(19) United States
(12) Patent Application Publication
(10) Pub. No.: US 2006/0199175 A1
(43) Pub. Date: Sep. 7, 2006

(54) Novel field testing method and device for detection of equine protozoal myeloencephalitis

(76) Inventors: Rama Sharma Kristipati, Fremont, CA (US); Tejinder Singh, Palo Alto, CA (US); Thelma H. Dunnebacke, Berkeley, CA (US); Anthony Caldwell, Frankfort, IN (US)

Correspondence Address: SHYAMALA T. RAJENDER, ESQ., SUITE 100, 4115 BLACKHAWK PLAZA CIRCLE, DANVILLE, CA, 94506, US

(21) Appl. No.: 11/072,337

(22) Filed: Mar. 7, 2005

(51) Int. Cl. C12Q 1/70; G01N 33/569; G01N 33/554; G01N 33/53

(52) U.S. Cl. 435/5; 435/7.22; 435/7.32

(57) ABSTRACT

The present invention discloses a quick, simple, qualitative and semi-quantitative immunologic method and device in a field test kit format for the detection and the stage of infection of certain protozoal, viral, bacterial, or nematodal pathogens in body fluids, using vaccines specific to such pathogens as antigens. The method comprises immobilizing an antigen specific to a selected protozoal, bacterial, viral or nematodal pathogen on a support membrane, obtaining a sample of a body fluid from a human or animal suspected of harboring the pathogen, thus containing antibodies thereto, contacting the antigen with the sample of body fluid and detecting the binding of the antigen with the antibodies by any suitable means. The device comprises a rigid member, an absorbent layer, a foam layer and a support member on which the antigen is immobilized, glued together to form a compact assembly. The field test kit comprises the device enclosed in a casing and vials containing additional reagents such as washing and dilution buffers, a chromogenic label for the detection of the antigen-antibody complex and positive and negative control solutions to verify the accuracy of the tests, instructions for use and the like. All the components are packaged in a small plastic container or box for easy transportation and use. While this invention is particularly adaptable to the detection of S. neurona using its vaccine as an antigen, other pathogens can also be detected by the method of this invention. These include but are not limited to Neospora hughesi, Neospora Canninum, Leishmania, Leptospira, West Nile virus, and other similar organisms.

### FIELD OF THE INVENTION

The present invention relates to a novel and quick method for the qualitative and semi-quantitative diagnostic detection of infectious agents and pathogens such as protozoa, viruses, nematodes, bacterial and other microorganisms, including protozoal parasites such as Sarcocystis neurona, usually abbreviated S. neurona, which cause equine myeloencephalitis, commonly known as EPM, and other pathogens which cause diseases such as Neospora hughesi, Neospora Canninum, Leishmania, Leptospira, West Nile virus, and other similar organisms and the like and a field test device therefor.

### BACKGROUND OF THE INVENTION

Diseases caused by protozoal parasites are one of the most difficult for medical science to understand and control effectively. Despite decades of research, significant understanding, diagnosis and treatment of the diseases have proved elusive. Vaccines have been developed against some of these pathogens and injected into the body of humans or animals to stimulate the production of antibodies to the pathogen which in turn will bind to the antigenic component of the pathogen thereby rendering it innocuous to the human or animal body.

A vaccine generally involves the use of dead, inactivated or weakened pathogen or its products, which are injected into the body of a human or animal to stimulate the production of protective antibodies without causing the disease. In other words, harmless or inactive antigens with epitopes which are found on the active pathogens are introduced into the body to stimulate production of antibodies specific to the epitopes of the pathogen. Various kinds of vaccines are produced and used in combating different pathogens, including bacteria, viruses, protozoal pathogens, nematodes and the like. Some of these vaccine types use: a) whole microorganisms killed and rendered harmless; or b) cultured organisms with reduced or attenuated pathogenicity; or c) toxoids or protein toxins released by the microorganism denatured in a manner that they still retain their epitopes or surface binding sites intact to elicit a protective reaction in the recipient animals or humans; or d) purified surface molecules which bind to the pathogens triggering destruction of the pathogen; or e) treated particles of the pathogen which retain the determinants or epitopes intact.

The production of any of these vaccines entails the identification, production, harvesting, purification and testing the efficacy of the vaccine against the targeted agent or pathogen. Some vaccines are active for the life of the animal or human while others are of shorter duration and have to be given periodically or in therapeutic situations. Vaccines so far have been used as preventive agents but have not been used in diagnostic tests.

Immunologic tests or immunoassays for the detection or diagnoses of the pathogens generally involve the production of antibodies to the pathogen, both polyclonal and monoclonal, isolating the antibodies, labeling the antibodies with an identifiable determinant, contacting the labeled antibody with a sample of the animal or human biological fluid such as blood, serum, plasma, urine, saliva etc., and testing for the formation or presence of an antibody-antigen complex formed, if the pathogenic antigen is present in the biological fluid. These methods are again, tedious, time consuming, require expert knowledge and training in immunochemical methods of analysis, such as the use of enzymes, radioisotopes or immuno-fluorescent markers as labels, washing off excess label and detecting and/or measuring the antibody-antigen complex formed.

A causative agent of equine protozoal myeloencephalitis, S. neurona, is an intracellular parasite that has a complex life cycle requiring two hosts, one a carnivore and one a herbivore. The protozoa have been found in various wild birds and small animals. The herbivores become infected when they ingest sporocysts of the protozoa in food and/or water contaminated by the fecal droppings of an infected carnivore. Meat eating animals become infected when they eat animals harboring sarcocysts of the protozoa. In a horse, infections have been attributed to the presence of opossums that leave infectious droppings in the pasture, hay, grain or water. A series of developmental stages leads to a migration pattern of the protozoa via blood from the intestine to neural tissues, where the protozoa divide in the cells of the brain and spinal cord resulting in the clinical symptoms of ataxia and focal muscular atrophy.

EPM, is thus a serious neurologic disease with enormous economic implications. The presence of the protozoa is usually identified during necropsy by the presence of neuronal lesions detected by microscopic examination of stained sections or by electron microscopy. Antibodies formed in response to the invasion of the protozoa have also been used to identify the presence of the protozoa and to determine their presence in body fluids such as blood, plasma, serum, cerebral spinal fluid, urine and saliva by various methods known in the art such as for example, indirect fluorescent antibody or Western blot analysis.

Diagnostic testing in live animals poses many practical problems and does not always yield reliable results. Existing tests based on Western Blot analysis require elaborate laboratory equipment and experienced technicians. They also require several days to perform and are expensive. Although some 50% of horses carry antibodies to S. neurona, thereby indicating that they have been exposed to the organism, few develop clinical symptoms and go undiagnosed. The cost of wide testing to detect such cases can be prohibitive.

Furthermore, some of these tests are not highly specific to the pathogen and may yield false negatives or false positives. Some of these prior art methods are described in the following reports, patents and trade journals.

“Piecing Together The EPM” The Horse Report, Vol. 21 (1), January 2003, Regents of The University of California, Davis, Calif., describes briefly the state of the art in diagnosing and treating equine EPM.

“Evaluation of New Antibody Tests for The Diagnosis of Equine Protozoal Myeloencephalitis (EPM)” Patricia A. Conrad, Bradd C. Barr, Ian A. Gardner and W. David Wilson, The 2003 Research Report, University of California, Davis, 2003, addresses the problems relating to the correct diagnosis and effective treatment of EPM in horses.

“Epidemiologic Studies of Transmission of Parasites Associated with Equine Protozoal Myeloencephalitis (EPM) on California Thoroughbred and Warmblood Farms” Ian A. Conrad, Bradd C. Barr, Patricia A. Conrad, Paulo C. Duarte and W. David Wilson, The 2003 Research Report, University of California, Davis, 2003, reports the results of research on the epidemiological studies on the transmission and serological patterns of the two pathogens involved in the clinical symptoms characteristic of EPM, namely, S. neurona, and Neospora hughesi to determine the pathogenesis and infective mechanisms of the disease and to help in the prevention, diagnosis and treatment of EPM.

“Helpful Tips” The Horse Report, Vol. 21 (1), University of California, Davis, January 2003, reports that current treatment of EPM using an anti-protozoal drug and the previous mode of treatment using a different anti-protozoal drug in combination with sulfonamide antimicrobial such as sulfadiazine with or without trimethoprim, are not very effective but seem to merely depress the parasite's viability such that the body's own immune system will have a chance to take over the control of the infection.

“Management of Equine Endoparasites” Valerie Wiebe, Animal Pharm News, University of California, Davis, April, 2004 reports a treatment protocol for EPM caused by S. neurona, the most common cause of EPM. The method consists of treating an infected animal with an antiprotozoal oral paste containing nitazoxianide. There are, however, severe adverse effects noted including deaths due to enterocolitis while administering the recommended dosages. Some of the adverse effects reported are fever, anorexia, lethargy, depression and laminitis in stallions.

U.S. Pat. No. 6,489,148 B1 issued Dec. 3, 2002 to Linda S. Mansfield, Alice J. Murphy and Mary G. Rossano, “Immunoassay For Equine Protozoal Myeloencephalitis in Horses” describes an immunoassay for S. neurona antibodies in horses using a Western blot method which again is time consuming, need long incubation times, laboratory equipment and trained personnel and cannot be readily performed in the field.

U.S. Pat. No. 6,344,337 B1 issued Feb. 5, 2002 to Linda S. Mansfield, Mary G. Rossano, Alice J. Murphy and Ruth A. Vrable, “Antigen Test To Detect Equine Protozoal Myeloencephalitis In Horse Serum And Cerebrospinal Fluid” is directed to an antigen-capture-based immunoassay for the detection of identifying antigens in horses infected with the EPM parasite, using monoclonal and polyclonal antibodies and employs an ELISA based or immunodiffusion-based assay for detecting the antibody-antigen conjugates. This method also requires laboratory equipment and trained personnel and takes many days before results can be obtained.

U.S. Pat. No. 6,153,394 issued Nov. 28, 2000, to Linda S. Mansfield, Alice J. Murphy and Mary G. Rossano, “Immunoassay For Equine Protozoal Myeloencephalitis In Horses” provides another immunoassay for the detection of S. neurona antibodies in horses which blocks certain nonspecific epitopes on the antigen to make the test more specific to antibodies to the pathogen. This assay uses a Western blot identification technique which requires long hours of incubation, electrophoresis, laboratory equipment and trained personnel and cannot be used in the field.

Similarly, many diseases caused by viruses and protozoal parasites have been particularly difficult to detect and diagnose early enough for timely treatment or require lengthy, time-consuming, expensive laboratory procedures. Some of these disease-causing organisms include but are not limited to S. neurona, Neospora hughesi, Neospora Canninum, Leishmania, Leptospira, and West Nile virus. There is no simple, quick field test for the early detection of any of these diseases. Of particular interest for the equine industry and for veterinarians is that there is no fast and simple diagnostic test currently available which can be used in the field by non-technical personnel for the detection of S. neurona antibodies in the body fluids of horses and ungulates.

The fast, simple diagnosis and timely treatment of EPM is of great economic importance and is of major concern to veterinarians and to the equine industry as well. If the diagnosis is delayed, or worse, if there is a misdiagnosis and the horse is not treated in a timely manner, the treatment which is highly expensive and labor intensive, may have to be prolonged and even with that, there may be permanent neurological damage and eventual death.

It is an object of the present invention, therefore, to provide a simple, fast diagnostic test to detect the presence of pathogens in the body fluids of an infected animal or human.

Another object of this invention is to provide a fast and simple method for the detection of several pathogens such as protozoa, bacteria, nematodes, viruses and the like, in a human or animal body by using vaccines against these pathogens as antigens and measuring the antibodies produced in reaction thereto.

Still another object is to measure the antibodies to a specific pathogen present in the body of an animal or human to detect the presence of the pathogen in the body.

Another object of the present invention is to provide a fast immuno-diagnostic test using a vaccine for a specific pathogen as an antigen to detect the presence of antibodies to that specific antigen.

Another object is to provide a simple, fast test for the detection of protozoa and viruses like S. neurona, Neospora hughesi, Neospora Canninum, Leishmania, Leptospira, and West Nile virus.

Still another object is to provide a simple, inexpensive test kit for the detection of EPM and other protozoal, bacterial, nematodal or viral diseases which can be used in the field by non-technical personnel.

Yet another object of the present invention is to provide a quick, easy and relatively inexpensive method for the testing of S. neurona in horses.

Another object of the present invention is to provide a method for the early detection of EPM.

Still another object is to provide a method whereby a distinction can be made whether the pathogenic infection in the animal or human is in the early stages or in later stages of manifestation.

Additional objects, advantages and novel features of the invention will be set forth in part in the description and drawings which follow, and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

To achieve the foregoing and other objects and in accordance with the purpose of the present invention as embodied and broadly described herein, the present invention is directed to a quick, inexpensive immuno-diagnostic method for the detection of a specific pathogen in body fluids of humans and animals.

In one specific embodiment, a method for the detection of antibodies to a pathogen in a body fluid, is provided where the method comprises providing an antigen to the antibodies; reacting the antigen with a sample of a body fluid of an animal or human containing said antibodies such that said antigen binds to said antibodies forming an antigen-antibody complex; reacting the antibody-antigen complex with a label such that it forms a readily detectible conjugate; and measuring the concentration of the conjugate as a measure of the concentration of the pathogen in the body fluid.

One embodiment of the present invention provides a simple membrane-capturing assay that requires no special instrumentation or training and can be used in the field to detect the presence of S. neurona induced antibodies in the blood sample within 3-5 minutes. The test utilizes antigens from killed S. neurona immobilized on a nitrocellulose membrane. The antigens are rendered non-infectious.

In another embodiment, the invention discloses a method whereby EPM or any other infectious disease resulting in antibodies or antigens in mammals or birds can be detected by the methods such as a flow through model; rapid lateral flow dipstick model; rapid lateral flow device model; ELISA based microtiter well model; and ELISA based micro tube model.

A preferred embodiment describes the flow through model of the instant method.

Yet another embodiment discloses a field test method for the detection of antibodies to S. neurona in equine serum or blood. A strip of nitrocellulose membrane is sandwiched between a lower absorbent layer of filter paper and a top layer of plastic foam in which a port or cutout is die-cut for the introduction of samples, reagents and wash buffers. A small sample of protein antibodies such as for instance, goat anti-mouse IgG, known to react nonspecifically with Protein A of the colloidal gold-Protein A conjugate, to yield a visible colored compound, is placed at a distinct spot on the nitrocellulose membrane assembly described. Excess IgG is washed off the nitrocellulose membrane with a suitable buffer. This serves as a control spot. A second small sample of the vaccine against S. neurona is also placed at another spot adjacent the IgG spot. This serves as the test spot. A serum or blood sample from a horse or other such animal, suspected of containing antibodies to S. neurona is obtained and contacted with the nitrocellulose membrane assembly with immobilized vaccine and the IgG. Unbound antibodies and excess serum are washed off with the buffer. The nitrocellulose layer is further contacted with a solution of colloidal gold conjugated with Protein A or Protein G to form a colored compound with the antigen-antibody complex and with the IgG. The intensity of the color of the compound at the test spot is determined as a indication of the concentration of the antibodies present in the body fluid of the horse or other animal. A serum sample from a healthy horse with no S. neurona induced antibodies serves as a negative control. The control test with IgG, and other tests with known samples for positive and negative results, establish the validity of the test and helps minimize false positives and false negatives.

In another embodiment, one control spot and two test spots are utilized. At the control spot, a protein antigen such as goat anti-mouse IgG is immobilized on the nitrocellulose membrane assembly. The two test spots contain the vaccine antigen for S. neurona. A serum or blood sample from a horse or other such animal, suspected of harboring S. neurona is obtained and contacted with the nitrocellulose membrane assembly with immobilized vaccine. Unbound antibodies and excess serum are washed off with the buffer. The nitrocellulose layer is further contacted with a solution of colloidal gold conjugated with Protein A or Protein G and a solution of colloidal gold conjugated with goat anti-equine Ig M and IgG, to form a colored compound with the antigen-antibody complex at the two test spots and with goat anti-mouse IgG at the control spot. The intensity of the color of the compounds at each test spot gives a measure of the concentration of S. neurona antibodies and if the infection is at an early stage or at a later stage, to determine the type of treatment needed. If the concentration of IgM, as reflected by the color intensity at one of the test spots, is found to be greater than that of the IgG, as reflected by the color intensity at the other test spot, it is an indication that the infection is at an early stage, the concentration of IgG increasing with the progression of the disease. A serum sample from a healthy horse with no S. neurona induced antibodies serves as a negative control.

In one specific embodiment, a portable test kit capable of operation in the field by non-technical personnel is also provided.

In another embodiment, the disclosed immunoassay test kit for detecting the presence of S. neurona induced antibodies in a sample of an equine body fluid, comprises an antigen specific to the antibodies, means for contacting the antigen with the body fluid sample, and means for detecting the binding of the antigen with the S. neurona induced antibodies.

In still another embodiment, a test kit for detecting the presence of antibodies to S. neurona in an equine subject is disclosed. The kit comprises a device containing a support member such as a nitrocellulose membrane on which an antigen to the antibodies is immobilized; an absorbent filter paper located below the nitrocellulose membrane; a top layer of plastic foam with a port or cutout for introducing body fluid samples, reagents, wash buffers and the like; and a label such as colloidal gold conjugated with Protein A or Protein G for the detection and measurement of the antigen-antibody complex.

In yet another embodiment of the present invention, the test device is constructed from a nitrocellulose member on which an antigen specific to the pathogen to be detected is immobilized. Preferably, the antigen is derived from a vaccine for the specific pathogen.

In still another embodiment of the present invention, the test device is a single use strip.

One embodiment of the present invention further includes method whereby blood, serum, saliva, tissue extracts, cerebrospinal fluid, urine and other body fluids can be tested for the presence of a particular pathogen.

In yet another embodiment, the pathogen is selected from a group consisting of S. neurona, Neospora hughesi, Neospora Canninum, Leishmania, Leptospira, and West Nile virus.

In a preferred embodiment of the present invention, the pathogen is S. neurona and the antigen is a vaccine therefor.

In another preferred embodiment a test kit is provided which contains other reagents used in the test.

Another embodiment provides a simple membrane-capturing assay for the presence of antibodies to a specific pathogen.

### BRIEF DESCRIPTION OF THE DRAWINGS

FIG. 1a is a top view of the device of the present invention. FIG. 1b is a bottom view of the same.

FIG. 2 is an illustration of the assembly of the device of the present invention.

FIG. 3 is a photograph of the test results.

FIG. 4 illustrates a plastic holder or casing for the test device and other components.

### DETAILED DESCRIPTION OF THE DRAWINGS

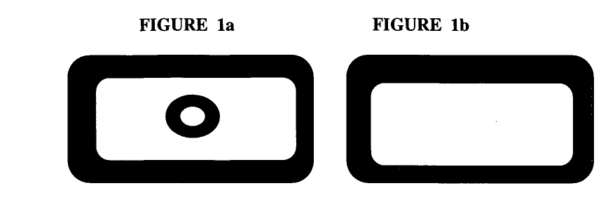

FIG. 1 shows the top and bottom views of the device of the present invention which comprises the bottom holder layer, the absorbent layer, the nitrocellulose or support layer and the top plastic foam layer with the center cutout which can be either circular, square, oval or rectangular. FIG. 1a shows the cutout in the top plastic foam layer. For purposes of illustration, the cutout is shown in a circular form.

FIG. 1b shows the bottom view of a paper, glass fiber, cardboard or similar material which gives the assembly the preferred rigidity and on to which is glued the absorbent filter paper layer.

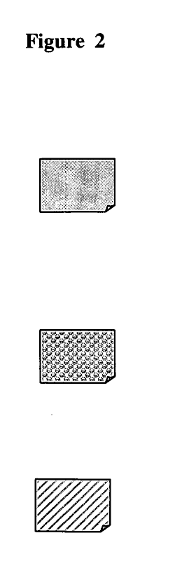

FIG. 2 depicts the assembly of the device. FIG. 2a illustrates the rigid glass fiber or cardboard member, cut to the desired size, as described in the description below. FIG. 2b illustrates the next layer which is an absorbent layer, preferably, filter paper which is glued on to the top portion of the rigid member. FIG. 2c shows the nitrocellulose support member on which the antigen is immobilized and is sandwiched between the absorbent filter paper layer and the top plastic foam layer illustrated in FIG. 2d. FIG. 2d illustrates the top plastic foam layer with a circular cutout for the introduction of the serum sample, wash buffers and other solutions. FIG. 2d also shows two circular spots where the control and test sample are placed on the nitrocellulose member through the cutout.

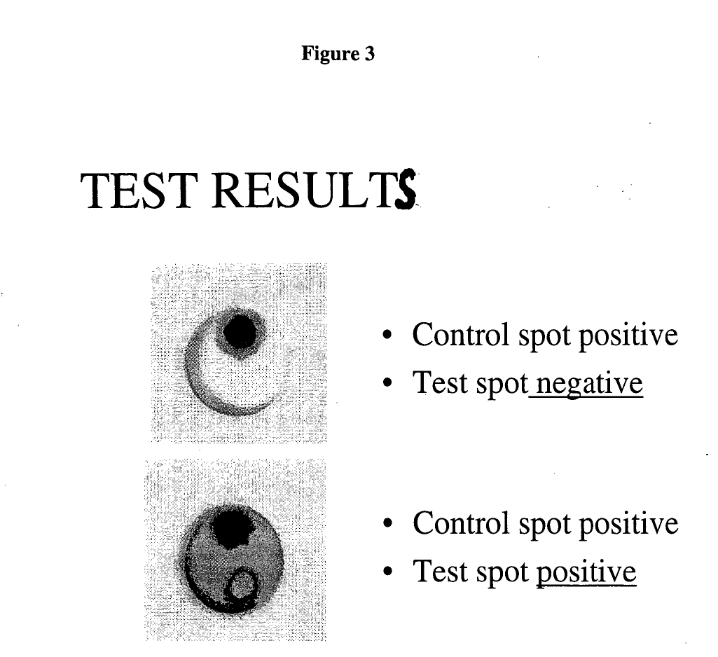

FIG. 3 illustrates the visual test results. FIG. 3a shows one red spot, which is the control spot and illustrates the non-specific binding of the IgG with the Protein A-gold conjugate. The test spot showed no visible reaction with the vaccine indicating that the serum sample did not contain S. neurona induced antibodies.

FIG. 3b shows two red spots, one a control spot with the goat anti-mouse IgG, and the other using the horse serum as the test sample. This is an indication that the animal tested positive for the presence of S. neurona induced antibodies. The intensity of the color at the test spot determines the level or concentration of the antibodies in the body fluid sample and is a measure of the progression of the infection.

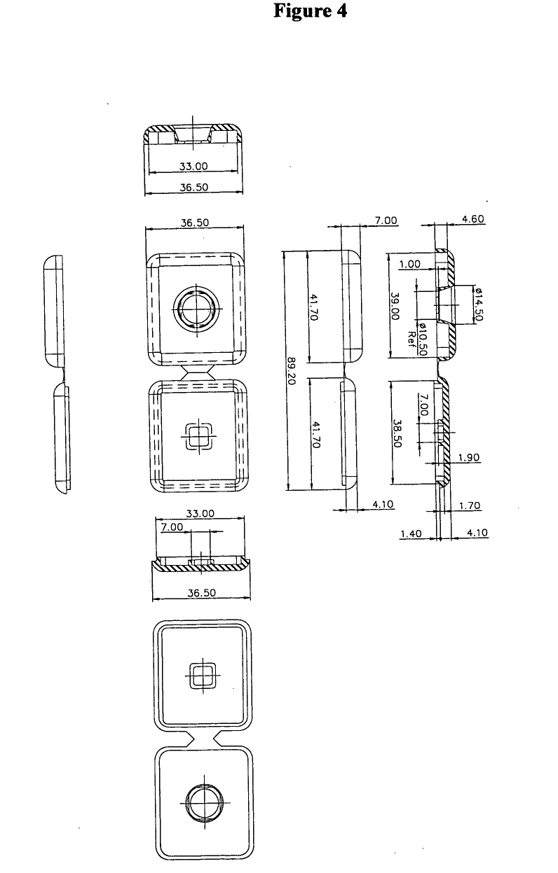

FIG. 4 shows the construction of the holder for the device of the present invention with the preferred dimensions and preferred materials to illustrate its portability and convenience of use. The holder is made of plastic or water-resistant cardboard or similar material and is of the same size as the device so the device fits snugly inside the plastic holder. The measurements shown are the preferred measurements and for illustration purposes only. Other suitable measurements and materials may be used.

### DETAILED DESCRIPTION OF THE INVENTION

The present invention provides a fast field test method for the diagnosis of many pathogens, specifically, protozoa, viruses, bacteria and nematodes and more specifically, S. neurona, which can be operated by someone with no special training or technical knowledge. The test method is a simple membrane-capturing assay that can be used in the field to detect the presence of S. neurona induced antibodies in body fluids such as blood, plasma, serum, urine, cerebrospinal fluid, tissue extracts and saliva, within about 3 to 5 minutes. One preferred embodiment of the invention is specifically directed to an immunologic method for the protozoa, S. neurona, by detecting antibodies present in the blood or serum of the infected animal, using a commercially available vaccine as the antigen therefor.

The present invention also provides a test device that utilizes antigens from killed S. neurona immobilized on a suitable support such as a nitrocellulose membrane. The antigens in the vaccine are rendered non-infectious. The method to detect antibodies or antigens in mammals or other animals can be used in various field or laboratory environments using various known techniques such as a flow through testing device; a rapid lateral flow dipstick type device; rapid lateral flow device; an ELISA based micro-titer well; or an ELISA based micro tube. Detection methodologies suitable for use with the present invention include a chromogenic reactions, Western blot, sandwich blot, ELISA, dot blot, slot blot, Northern blot and antibody precipitation.

The device and method for the detection of a specific pathogen, such as S. neurona, in body fluids of an infected animal, using an antigen such as a vaccine against S. neurona, are described in detail below. A support member such as a nitrocellulose membrane, preferably with a filter paper backing to give it rigid support, is cut to desired dimensions, and placed on top of an absorbent layer such as filter paper cut to the same size as the nitrocellulose member. A plastic foam member or strip is cut to the same size as the nitrocellulose member, and a central port or cutout is then die-cut in the foam layer for the introduction of the sample serum, wash buffers, gold conjugate and other reagents. For purposes of illustration, the central port is shown as a circle but it can be a square, rectangle, ellipse or any other suitable shape.

A strip of an absorbent pad such as filter paper or other absorbent material, 1.5 to 2.0 mm thick, is cut to the same size as the nitrocellulose member, to absorb the wash buffers, excess serum and other materials washed off the surface of the nitrocellulose members. The nitrocellulose membrane is then sandwiched between the absorbent layer and the foam layer and glued to both to hold it in place. A small sample of an antigen, such as a vaccine against S. neurona, containing from 50 to 1000 ng of protein, is spotted on the nitrocellulose assembly and immobilized on the nitrocellulose membrane. A control spot consisting of an animal immunoglobulin which includes but is not limited to goat IgG, also containing from 50 to 1000 ng of protein, and known to bind nonspecifically with Protein A of the gold conjugate used as a label, is also placed on the nitrocellulose assembly at a spot adjacent to the test spot (see FIG. 3d) and allowed to dry. The nitrocellulose membrane assembly containing the control spot and the test spot is allowed to dry overnight at room temperature. The nitrocellulose membrane assembly is then cut to the desired size and placed in a plastic casing or cassette for easy transportation and to prevent contamination. The casing is preferably made from high-density polycarbonate, but similar materials which include but are not limited to polypropylene, low-density polycarbonate, polystyrene, plastic, metal, and paper may also be used. This assembly is defined as the test device of this invention and is referred to as such in this description.

The present invention additionally discloses a field test kit which includes a single or plurality of test devices described above, ranging from one to twenty five, preferably five and still more preferably two for convenience of packing and shipping, individually pouched and sealed, two or three dropper pipets, a bag of desiccant to keep the components dry, vials of buffers containing phosphate buffered saline, bovine serum albumin, and Tween 20, vials of gold conjugated Protein A or Protein G, a pair of gloves and a plastic disposal bag. All these components are contained in a plastic box or container. The box or container may also be constructed out of cardboard, metals such as aluminum, tin, zinc and the like, glass, or polycarbonate and the like. The kit may also additionally include vials of goat anti-equine IgG and IgM, or mouse anti-equine monoclonal antibodies, to measure the progression or the stage of S. neurona infection in the animal.

When the test kit is used for measuring the progression of the EPM disease, the gold is conjugated with goat anti-equine IgM or IgG antibodies or with mouse anti-equine monoclonal antibodies. There are two test spots, one for testing using IgG conjugated label and the other using IgM conjugated label.

The invention also discloses a method for the detection as well as the progression of S. neurona infection in horses. A method comprises providing a nitrocellulose membrane assembly, consisting, as described herein, of a layer of a nitrocellulose membrane sandwiched between a layer of a thick filter paper and a plastic foam layer with a central cutout portion for the introduction of samples, reagents, buffers and the like. A sample of a vaccine antigen against S. neurona is then immobilized on the nitrocellulose membrane assembly at two discrete spots thereon, appropriately identified. These serve as two separate test spots. The excess vaccine is washed off the nitrocellulose membrane with a suitable buffer such as PBS-BSA-Triton buffer. A protein immunoglobulin such as goat anti-mouse IgG, known to react with a known label such as colloidal gold conjugated with Protein A or goat anti-equine IgG or IgM, or mouse anti-equine monoclonal antibodies, is also immobilized on the nitrocellulose membrane at a spot adjacent the two test spots. This serves as control spot. The excess protein immunoglobulin is washed off the nitrocellulose membrane. A serum sample from a horse suspected of harboring said S. neurona is contacted with the vaccine antigen immobilized on the nitrocellulose membrane. Unbound antibodies and excess serum are then washed off the nitrocellulose membrane. The membrane is then contacted with a label comprising a solution of colloidal gold conjugated with goat anti-equine IgG and IgM. The IgG and IgM, and colloidal gold bind with the antigen-antibody complex to form a colored product. The intensity of color at the two test spots and thus the concentration of the antibodies is determined by visual inspection or by a spectrophotometer in a laboratory. The IgM component of the label binds more strongly when the S. neurona infection is in the early stages and the IgG component of the label binds more strongly as the infection and the disease progresses. Determining their relative concentrations at the two discrete test spots gives an indication of the stage of S. neurona infection.

The following examples illustrate the method of the invention, the construction of the device and the field test kit. They are presented for purposes of illustration only and are not intended to limit the invention to the specific embodiments or methods discussed in detail in these examples and illustrated in the drawings.

Assembly of the Device

The construction of the membrane of the present invention comprised the steps of (a) obtaining a specially designed and die-cut plastic foam tape, (b) cutting it to a size of from about 9 mm to about 14 mm long, preferably to about 11.5 mm length, with a central circular cutout of from about 8 mm to about 12 mm, preferably 9 mm diameter; (c) coating of the backing of the foam tape with a medical grade gum, (d) obtaining a nitrocellulose or a similar material with a filter paper backing to give rigid support to the fragile nitrocellulose membrane, (e) cutting the nitrocellulose membrane to cover the entire central circle, and (f) placing on the nitrocellulose members the desired antigens or antibodies or any suitable proteins and peptides as a single spot or multiple spots. The foam cutout in step (a) may be circular, oval, elliptical, rectangular or square, the purpose being to retain the added reagents within the boundary of the cutout thereby allowing the reagents to sink through the nitrocellulose membrane and absorbed by the filter paper backing.

The nitrocellulose membrane used in the present device was selected with a preferred pore size of 0.45 μm but membranes with different pore sizes of from about 0.2 μm to about 8 μm may be used. The nitrocellulose membrane may be used with or without a filter paper backing. The absorbent pad about 1.5 to 2.0 mm thick, was cut to a specific width from about 0.5 in to about 2 in. This pad was positioned below the nitrocellulose layer, sandwiching the nitrocellulose between it and the foam layer. The purpose of this absorbent pad was to absorb all the reagent solutions passing through the nitrocellulose layer.

All the layers are glued together to form a compact membrane assembly and is referred to as the device of this invention.

Testing for the Presence of S. neurona Antibodies in Horses

Antibodies to S. neurona present in the body fluids of an infected animal, such as a horse, were captured by S. neurona antigens immobilized in a designated spot on a nitrocellulose membrane. As a control, a second designated spot of non-specific IgG (immunoglobulins) was added to the membrane. The membrane with the bound antibodies was then exposed to colloidal gold or dye particles, coupled to affinity purified goat anti-equine IgG, IgM, Protein A or Protein G. The nitrocellulose membrane was washed with 3-4 additions of 150 μl buffer to wash away unbound proteins. A resulting single red spot on the membrane indicated that the test was valid, and the presence of only one red spot indicating the absence of S. neurona antibodies. That was a negative test for S. neurons. The presence of a second red spot on the membrane, a spot over the region containing the antigen from the vaccine, signaled the immunologic binding of the antigen with S. neurona antibodies in the body fluids. That indicated a positive test for S. neurona.

Blood Sample Collection

All the test samples were collected aseptically by venipuncture of test horses and placed into heparinized tubes. These were inverted 2-3 times to allow separation into blood cell sediment and clear plasma or serum. All the test samples employed here were obtained from Indiana Horse Rescue Center. Equine sera used for negative controls, bovine sera and human sera were purchased from a commercial source. All test samples were stored at 4-8° C.

Device Casing

The device was placed in a casing made according to the specifications described in FIG. 4. The casing was made using high-density polycarbonate. The device casing can also be made out of similar materials like polypropylene, low-density polycarbonate, polystyrene, or suitable combinations of plastics, metals, paper or rigid paper alone.

Antigen Preparation

S. neurona vaccine (a formulated solution containing killed protozoa) was obtained from a commercial supplier in 10 mL vials ready to use as a vaccine. This vaccine preparation was purified and characterized before use. The vaccine in the vials was then subjected to simple denaturing by repeated thawing (room temperature 25-28° C.) and freezing (−20° C.). This process was repeated at least 5 times. A small amount of about 0.5 ml of this solution was pipetted into 0.5 ml Eppendorf tubes containing a 10 thousand molecular weight cutoff separation membrane and were centrifuged at 13 thousand rpm for 5-10 minutes. The supernatant retaining the higher molecular weight proteins i.e. above 10 thousand cutoff, was mixed with cold phosphate buffered saline and the process of centrifuging the vaccine was repeated 4-5 times until a clear solution was obtained. The protein content in the modified preparation was determined by measuring the optical density at 280 nm using a spectrophotometer. This protein purification/concentration process can be scaled up for larger volumes of vaccine antigen.

Antigen Coating on Nitrocellulose Membrane

A small volume of the EPM vaccine antigen processed as described above was mixed with a 3-5% by volume solution of sucrose and a 0.05% by volume solution of sodium azide. Both chemicals were obtained commercially. A small quantity, about 0.5 μl of this vaccine preparation containing 50-1000 ng of protein was spotted onto the nitrocellulose membrane. A control spot of about 50-1000 ng in 0.5 μl consisting of animal immunoglobulins (IgG or IgM) preparation, such as goat IgG or IgM obtained from commercial suppliers, was also applied to the membrane. The nitrocellulose membrane assembly containing the control spot and test spot was allowed to dry overnight at room temperature (25-28° C.). The membrane assembly was cut to the appropriate size and placed in the plastic casing. The completely assembled devices were stored at room temperature (25-28° C.) in a drum containing a desiccant. The shelf life of the test device of this invention is at least one year without any significant loss of potency.

Wash Buffer

The wash buffer contained phosphate buffered saline with 0.1% Tween-20 and 0.25% Bovine Serum Albumin (PBSTB). The purpose of washing the membrane with this buffer was to block nonspecific binding on the membrane and wash away unbound antibodies and proteins.

Preparation of Gold Labeled Protein A

The detection of antibodies present in the test sample, after binding to S. neurona vaccine antigen in the device, was achieved using colloidal gold conjugated with Protein A. This is sometimes referred to as the gold activator as it promotes the color reaction. Although Protein A was used in these examples, other gold preparations with protein G or mouse anti-equine IgG or IgM monoclonal or polyclonal antibodies can also be used. The usage of other materials like magnetic particles, polystyrene, nylon, glass beads and carbon particles for protein, antibody and antigen conjugation are also within the scope of this invention. The protein A-gold solution was diluted with PBSTB to give a concentration of one optical density unit at 530 nm.

Controls for Tests

Equine serum obtained from a commercial source showed no reaction in this EPM testing procedure and the instant EPM test. The batch of serum was purchased in a large volume and kept for further use as our negative control serum.

Anti-rabbit S. neurona antibodies raised in rabbits served as positive controls.

Testing Procedure

The testing protocol used was as follows:

A blood sample was obtained from an animal in a heparinized collection tube, using aseptic techniques as described in a preceding section. The separated serum was extracted into a syringe. Two drops of wash buffer were applied to the test well in the test device, allowing each drop to soak in. Two drops of serum sample were then applied to the test well, allowing each drop to soak in. Another two drops of buffer were applied to the test well, allowing each drop to soak in. Two drops of the gold conjugate activator were applied to the test well. Two drops of wash buffer were applied to the test well. Test results reached a steady state within 3 minutes.

When the reading reached a stable state, the control spot appeared as a red or dark pink spot as it should. This served as a validation. The test spot should be colorless if the test is negative, i.e., the serum or plasma does not contain antibodies to S. neurona. If the test spot is pink to red in color, the test is positive for the presence of antibodies to S. neurona in the test sample. The darker the red-color, the greater quantity of antibodies to S. neurona; this indicates a higher level of antibodies, and thus, a higher progression of the disease.

Packaging the EPM Test Kit

The packages of the EPM test kit vary in the number of devices included therein depending on the number of tests desired to be performed in the field and ranged from two to twenty five. A typical package contains two individually pouched and sealed test devices, two dropper pipettes (for addition of serum) and a bag of desiccant to keep the contents dry. The reagents are in small dropper bottles for convenience including Gold conjugated Protein A or Protein G (0.5 ml, red cap bottle) and wash buffer (1.5 ml, white cap bottle), a pair of gloves and a bag for disposal. All of these components along with an instruction sheet are packed in a box with appropriate labels. Test kits for testing the progression of EPM with time included separate vials of colloidal gold conjugated with goat anti-equine IgG and Gold conjugated with goat anti-equine IgM or gold conjugated with mouse anti-equine monoclonal antibodies. All the dropper bottles, pouches, dropper pipettes, and desiccant materials were purchased from commercial suppliers.

To test the usefulness of the method and kit of the present invention, blood samples from 25 horses with various clinical symptoms were collected. The blood was drawn into heparinized vacuum tubes. The tubes were kept refrigerated until they were used for testing using the kit of the invention. Some of the samples were tested by the standard Western Blot (WB) test for confirmation of the results obtained with the method of the present invention. A selection of the samples were also subjected to the Immun-fluorescent Antibody test (IFA), a test that is considered the gold standard for identifying the presence of the protozoa S. neurona. The results of all these tests are given in the following chart.

TABLE 1A
Summary Of Test Results With the Test Kit of the Present Invention

| | Test results with EPM | |
|---|---|---|
| Sample | Check | Quantitative Value |
| Horse # 1 | Strong positive | +4 WB positive; IFA positive |
| Horse # 2 | Weak positive | +1 |
| Horse # 3 | Negative | 0 |
| Horse # 4 | Negative | 0 |
| Horse # 5 | Weak positive | +1 |
| Horse # 6 | Intermediate positive | +2 |
| Horse # 7 | Intermediate positive | +2 |
| Horse # 8 | Weak positive | +1 |
| Horse # 9 | Negative | 0 |
| Horse # 10 | Negative | 0 |
| Horse # 11 | Strong positive | +3 WB positive; IFA positive |
| Horse # 12 | Weak positive | +1 IFA positive |
| Horse # 13 | Intermediate positive | +2 |
| Horse # 14 | Intermediate positive | +2 |
| Horse # 15 | Intermediate positive | +2 |
| Horse # 16 | Intermediate positive | +2 |
| Horse # 17 | Weak positive | +1 |
| Horse # 18 | Strong positive | +3 WB positive; IFA positive |
| Horse # 19 | Weak positive | +1 |
| Horse # 20 | Strong positive | +3 |
| Horse # 21 | Intermediate positive | +2 IFA positive |
| Horse # 22 | Intermediate positive | +2 |
| Horse # 23 | Strong positive | +3 |
| Horse # 24 | Intermediate positive | +2 |
| Horse # 25 | Weak positive | +1 |

0 = negative;
+1 = weak positive;
+2 = intermediate positive;
+3 & +4 = strong positive
* Positive using Western Blot Test;
** Positive using immuno-fluorescent antibody test

TABLE 1B
Commercially Available Sera For Specificity Testing

| Sample No. | Test results | Quantitative Value |
|---|---|---|
| Bovine # 1 | Negative | 0 |
| Bovine # 2 | Negative | 0 |
| Bovine # 3 | Negative | 0 |
| Human # 1 | Negative | 0 |
| Human # 2 | Negative | 0 |
| Equine # 1 | Negative | 0 |
| Equine # 2 | Negative | 0 |
| Equine # 11 | Strong Positive | +3 |

B = Bovine serum;
H = Human serum;
E = Horse serum
Equine # 1 was negative using immuno-flourescent Antibody test

In all cases the EPM antibody field test results were consistent with the results from Western Blot serum analysis when available. The positive field tests were consistent with the clinical symptoms displayed by the animals.

TABLE IC
Summary Of Field Test Results Using The Present Test Kit

| Sample Nos. | Test results with subject kit | Quantitative Value |
|---|---|---|
| 6 horses | Strong positive | +3 or +4 |
| 16 horses | Intermediate positive | +2 |
| 20 horses | Weak positive | +1 |
| 14 horses | Negative | 0 - IFA negative |

In the field test examples described above, the preparations using a vaccine antigen, have shown consistency and specificity in each of the test formats used. Blood samples from horses that tested positive by the procedure of the present invention also tested positive by Western Blot analysis, and by the immuno-fluorescence antibody assay. Additionally, positive tests obtained using antisera of three rabbits that had been inoculated with S. neurona verified that the antigen used in the test was a component of S. neurona. Negative blood samples in the included in the tests were known not to be infected with S. neurona protozoa and were samples of “normal” equine, bovine and human blood.

Other organisms detected by the use of the detailed procedures described above include but are not limited to Neospora hughesi, West Nile virus, Leishmania, Leptospira, and the like, for which vaccines containing whole or portions of the pathogenic organisms are available.

While the present method and kit allow for early detection of the debilitating and frequently fatal disease EPM, it can also be used for monitoring of the antibody levels against any other protozoal, bacterial nematodal or viral pathogens. The ease of the test is obvious and the risk is no greater than animals receiving annual vaccinations. The potential cost savings to the equine owner are approximately 75% less than a Western blot serum analysis and results are available in the field within minutes.

The foregoing description of the preferred embodiments of the subject invention have been presented for purposes of illustration and description and for a better understanding of the invention. It is not intended to be exhaustive or to limit the invention to the precise form disclosed; and obviously many modifications and variations are possible in light of the above teaching. The particular embodiments were chosen and described in some detail to best explain the principles of the invention and its practical application to thereby enable others skilled in the relevant art to best utilize the invention in various embodiments and with various modification as are suited to the particular use contemplated. It is intended that the invention be defined by the claims appended hereto.

What is claimed is:

1. A method for the detection of antibodies to a pathogen in a body fluid, said method comprising the steps of
a) providing an antigen to said antibodies;
b) contacting said antigen with said body fluid containing said antibodies such that said antigen binds to said antibodies forming an antigen-antibody complex;
c) providing a means which reacts with said antibody-antigen complex to form a readily detectible conjugate; and
d) providing means to measure the concentration of said conjugate as a measure of the concentration of said pathogen in said body fluid.

2. The method of claim 1, wherein said antigen is a vaccine against said pathogen.

3. The method of claim 2, wherein said antigen is an antigen specific to said pathogen and is extracted from said vaccine.

4. The method of claim 1, wherein said pathogen is selected from the group consisting of S. neurona, Neospora Lughesi, Neospora Canninum, Leishmania, Leptospira, and West Nile Virus.

5. The method of claim 4, wherein said pathogen is S. neurona.

6. The method of claim 5, wherein said body fluid is selected from the group consisting of cells, whole blood, plasma, serum, saliva, tissue extract, urine and cerebrospinal fluid.

7. The method of claim 6, wherein said body fluid is serum.

8. The method of claim 6, wherein said body fluid is plasma.

9. The method of claim 6, wherein said body fluid is whole blood.

10. The method of claim 6, wherein said body fluid is cerebrospinal fluid.

11. The method of claim 5, wherein said antigen is immobilized on a support medium.

12. The method claim 11, wherein said support medium is nitrocellulose or polyvinylidine fluoride.

13. The method of claim 12, wherein said support medium is nitrocellulose.

14. The method of claim 13, wherein said means which reacts with said antigen-antibody complex is a chromogen.

15. The method of claim 14, wherein said chromogen is colloidal gold.

16. The method of claim 15, wherein said gold is conjugated with Protein A or Protein G.

17. The method of claim 15, wherein said gold is conjugated with goat anti-equine IgG or IgM.

18. The method of claim 17, wherein said gold is conjugated with goat anti-equine IgG.

19. The method of claim 17, wherein said gold is conjugated with goat anti-equine IgM.

20. The method of claim 15, wherein said gold is conjugated with mouse anti-equine monoclonal antibodies.

21. The method of claim 16, wherein said gold is conjugated with Protein A.

22. The method of claim 1, wherein said means of measuring the concentration of said conjugate is selected from the group consisting of a chromogenic reaction, Western blot, sandwich blot, ELISA, dot blot, slot blot, Northern blot and antibody precipitation.

22. The method of claim 21, wherein said means of measuring the concentration of said conjugate is a chromogenic reaction.

23. The method of claim 22, wherein said color is detected by visual inspection or measured by photometric analysis.

24. The method of claim 23, wherein said color is detected by visual inspection.

25. The method of claim 11, wherein said vaccine is whole attenuated organism or whole killed organism.

26. The method of claim 25, wherein said vaccine is whole killed organisms of the S. neurona virus.

27. The method of claim 25, wherein said vaccine is whole attenuated organisms of the S. neurona virus.

28. The method of claim 11, wherein said antigen is a protein purified from said vaccine.

29. A method for the detection of antibodies to S. neurona in the body fluid of an animal comprising the steps of a) providing an antigen to said antibodies immobilized on a support medium; b) contacting said antigen with said body fluid containing said antibodies so that said antibodies form an insoluble, readily detectible complex with the antibodies; and d) providing means to measure the concentration of said complex as a measure of the concentration of said S. neurona in said body fluid.

30. The method of claim 29, wherein said antigen is a vaccine for said S. neurona.

31. The method of claim 30, wherein said antigen is an antigen specific to S. neurona and is extracted from said vaccine.

32. The method of claim 31, wherein said body fluid is selected from the group consisting of cells, whole blood, plasma, serum, saliva, tissue extract, urine and cerebrospinal fluid.

33. The method of claim 32, wherein said body fluid is serum.

34. The method of claim 32, wherein said body fluid is plasma.

35. The method of claim 32, wherein said body fluid is whole blood.

36. The method of claim 35, wherein said animal is a horse.

37. The method of claim 36, wherein said support medium for said antigen is nitrocellulose.

38. The method of claim 29 wherein said detection means further comprises providing colloidal gold conjugated with Protein A and contacting said gold conjugate with the antigen-antibody complex to form a color reaction; and measuring the color intensity as a measure of the level of said antibodies in said body fluid.

39. The method of claim 29 wherein said detection means further comprises providing colloidal gold conjugated with goat anti-equine IgG or IgM and contacting said gold conjugate with the antigen-antibody complex to form a color reaction; and measuring the color intensity as a measure of the level of said antibodies in said body fluid.

40. The method of claim 39 wherein said detection means further comprises providing colloidal gold conjugated with goat anti-equine IgG and contacting said gold conjugate with the antigen-antibody complex to form a color reaction; and measuring the color intensity as a measure of the level of said antibodies in said body fluid.

41. The method of claim 39 wherein said detection means further comprises providing colloidal gold conjugated with goat anti-equine IgM and contacting said gold conjugate with the antigen-antibody complex to form a color reaction; and measuring the color intensity as a measure of the level of said antibodies in said body fluid.

42. The method of claim 39 wherein said detection means further comprises providing colloidal gold conjugated with goat anti-equine IgG and IgM and contacting said gold conjugate with the antigen-antibody complex to form a color reaction; and measuring the color intensity as a measure of the level of said antibodies in said body fluid.

43. In a method for the detection of antibodies to S. neurona in the body fluid of a horse, comprising the steps of a) providing a support medium on which an antigen to said antibody is immobilized; b) contacting said antigen with said body fluid containing said antibodies such that said antibody forms an insoluble, readily detectible complex; and d) providing means to measure the concentration of said conjugate as a measure of the concentration of said S. neurona, the improvement wherein said antigen is a vaccine for said S.neurona.

44. The method of claim 43, wherein said body fluid is selected from the group consisting of cells, whole blood, plasma, serum, saliva, urine, tissue extract and cerebrospinal fluid.

45. The method of claim 44, wherein said body fluid is serum.

46. The method of claim 44, wherein said body fluid is plasma.

47. The method of claim 44, wherein said body fluid is whole blood.

48. A method for making an immunoassay test kit for S. neurona induced antibodies comprising the steps of
a) providing an antigen specific to said S. neurona induced antibodies;
b) immobilizing said antigen on a support means;
c) providing means for contacting said antigen with a sample of a body fluid of an animal suspected of harboring said S. neurona;
d) providing an indicator means for measuring the binding of said antigen with said antibodies; and
e) providing means for determining the concentration of said binding of said antigen with said antibodies.

49. The method of claim 46, wherein said antigen, immobilizing support means, said means for contacting said antigen with said sample and said indicator means for measuring the binding of said antigen with said sample are assembled in a casing.

50. The method of claim 48 wherein the components are assembled into a dipstick or cartridge format.

51. The method of claim 49 further comprising providing instructions for use of said kit and assembling said instructions together with said antigen, support means, and indicator means in said casing.

52. The method of claim 51 wherein said indicator means is colloidal gold.

53. A method of detecting the presence of S. neurona in a body fluid of an ungulate or other ruminant animal comprising the steps of
a) obtaining a sample of a body fluid from said animal;
b) contacting said body fluid with an antigen specific to S. neurona;
c) providing a detectible label which binds to the antigen-antibody complex; and
d) detecting the presence of said antigen-antibody complex to indicate the presence of S. neurona in said sample.

54. A field test method for the detection of antibodies to S. neurona in equine serum, said method comprising the steps of
a) obtaining a sample of a vaccine against S. neurona;
b) immobilizing said vaccine on a nitrocellulose membrane at a discrete spot thereon;
c) washing off excess vaccine with PBS-BSA-Triton buffer;
d) obtaining a sample of a protein immunoglobulin known to react with a known label;
e) immobilizing said protein immunoglobulin sample on said nitrocellulose membrane;
f) washing off excess protein immunoglobulin from said nitrocellulose membrane;
g) obtaining a serum sample from a horse suspected of harboring said S. neurona;
i) contacting said nitrocellulose membrane with said serum sample;
j) washing off unbound antibodies and excess serum from said nitrocellulose membrane;
k) further contacting said nitrocellulose membrane with said known label comprising a solution of colloidal gold conjugated with Protein A;
l) determining the intensity of the color of the antigen-antibody-label complex developed as a measure of the concentration of S. neurona induced antibodies in said serum sample.

55. A method for testing the presence of antibodies to a pathogen in a sample comprising providing an immunoassay test device for detecting the presence or level of said antibody in a sample, said device further comprising an antigen specific to said antibody, a support for said antigen, means for contacting said antigen with said sample, and a means for detecting binding of said antibody with said selected antigen.

56. The method of claim 55 wherein said means for detecting the presence of a selected antibody is selected from the group consisting of a chromogenic reaction, Western blot, sandwich blot, ELISA, dot blot, slot blot, Northern blot, and antibody precipitation.

57. The method of claim 55 wherein the level of said selected antibody indicating the animal is infected with the S. neurona protozoa is substantially above the background level of said selected antibody in a non-infected animal.

58. The method of claim 55 wherein said sample is selected from a group consisting of cells, whole blood, plasma, urine, tissue extract, saliva, cerebrospinal fluid and serum.

59. The method of claim 58 wherein said sample is serum.

60. The method of claim 58 wherein said sample is whole blood.

61. The method of claim 60 further comprising filtering said blood sample to obtain serum containing said antibodies.

62. A device for the detection of antibodies to a pathogen in a body fluid, said device comprising
a) a support member on which an antigen specific to said antibodies is immobilized;
b) an absorbent second member located beneath said support member;
c) means for contacting said antigen with said body fluid containing said antibodies to form an antigen-antibody complex;
d) means for contacting said complex with a label which forms an insoluble, readily visible colored compound; and
e) means for measuring the intensity of said compound as a measure of the concentration of said pathogen in said body fluid.

63. The device of claim 62 wherein said antigen is a vaccine against S. neurona.

64. The device of claim 63 said support member on which said vaccine is immobilized is nitrocellulose or polyvinylidine.

65. The device of claim 64 wherein said support member is a nitrocellulose.

66. The device of claim 65 wherein said second absorbent member is filter paper.

67. The device of claim 66 wherein said means for contacting said vaccine with said body fluid is a layer of plastic foam with a center cutout portion placed on top of said nitrocellulose membrane.

68. The device of claim 67 further comprising an additional rigid support member placed beneath said filter paper layer.

69. The device of claim 67 wherein said additional rigid support member, said filter paper layer, said nitrocellulose membrane and said plastic foam layer with a center cutout are glued together to form a compact membrane assembly.

70. The device of claim 69 wherein said assembly is contained in a casing.

70. The device of claim 69 wherein said assembly is contained in a casing.

71. The device of claim 70 wherein said casing is constructed from a material selected from a group consisting of high-density polycarbonate, polypropylene, low-density polycarbonate, polystyrene, plastic, metal, and paper.

72. The device of claim 71 wherein said casing is constructed from high-density polycarbonate.

73. An immunoassay test kit for detecting the presence of S. neurona induced antibodies in a sample of an equine body fluid, said kit comprising a device according to claim 72 for immobilizing an antigen specific to said antibodies on a support means, means for contacting said immobilized antigen with said sample and means for detecting the binding of said antigen with said antibodies in said sample.

74. The kit of claim 73, wherein said body fluid is selected from a group consisting of whole blood, serum, plasma, cells, saliva, tissue extract, urine and cerebrospinal fluid.

75. The kit of claim 74 wherein said body fluid is serum.

76. The kit of claim 74 wherein said body fluid is plasma.

77. The kit of claim of 74 wherein said body fluid is whole blood.

78. The kit of claim 74 additionally including vials of buffers, positive and negative controls, gold conjugates with protein A and protein G and instructions for use.

79. The kit of claim 78, further including gold conjugates with goat anti-equine IgG and IgM.

80. The kit of claim 78, further including gold conjugates with mouse anti-equine monoclonal antibodies.

81. The kit of claim 78 additionally including a plurality of said devices, dropper pipettes, desiccant, and hand gloves.

82. In a method for the detection of the presence of antibodies to S. neurona in horses, the improvement comprising:
using a vaccine against said S. neurona as the antigen for binding to said antibodies and detecting the level of antigen-antibody binding as a measure of the level of S. neurona present in said horse.

83. A method for the detection and progression of S. neurona infection in horses said method comprising the steps of
a) providing a nitrocellulose membrane assembly according to claim 69;
b) obtaining a sample of a vaccine antigen against S. neurona;
c) immobilizing said vaccine on said membrane assembly at a discrete spot thereon as a test spot;
d) washing off excess vaccine with PBS-BSA-Tween 20 buffer;
e) obtaining a sample of a protein immunoglobulin known to react with a known label;
f) immobilizing said protein immunoglobulin sample as a control spot on said nitrocellulose membrane at a spot adjacent to said test spot;
g) washing off excess protein immunoglobulin from said nitrocellulose membrane;
h) obtaining a serum sample from a horse suspected of harboring said S. neurona;
i) contacting said nitrocellulose membrane with said serum sample;
j) washing off unbound antibodies and excess serum from said nitrocellulose membrane;
k) further contacting said nitrocellulose membrane with said label comprising a solution of colloidal gold conjugated with goat anti-equine IgG and IgM;
l) determining the intensities of the color of the antigen-antibody-IgG-label complex and antigen-antibody-IgM-label complex developed as a measure of the concentration of S. neurona induced antibodies in said serum sample and the stage of infection.

84. The method of claim 83, wherein said label is a solution of colloidal gold conjugated with mouse anti-equine monoclonal antibodies.

85. The method of claim 83, wherein said test spot in step c) comprises two test spots.
